(19) United States
(12) Patent Application Publication
(10) Pub. No.: US 2026/0308693 A1
(43) Pub. Date: Oct. 8, 2026

(54) METHODS AND SYSTEMS FOR DETECTING A PRIMARY DISEASE

(71) Applicant: Inivata Ltd., Cambridge (GB)

(72) Inventors: Tauanne Dias Amarante, Cambridge (GB); Tim Forshew, Hitchin (GB)

(21) Appl. No.: 19/548,580

(22) Filed: Feb. 24, 2026

Related U.S. Application Data
(63) Continuation-in-part of application No. 19/269,949, filed on Jul. 15, 2025.
Application No. 19/269,949 is a continuation of application No. PCT/IB2024/000023, filed on Jan. 17, 2024.
(60) Provisional application No. 63/439,769, filed on Jan. 18, 2023.

(51) Int. Cl. C12Q 1/6886

(52) U.S. Cl. CPC C12Q 1/6886; C12Q 2600/154; C12Q 2600/156

(57) ABSTRACT

Provided, among other things, is a method of detecting a presence or absence of a second primary disease in a subject. In some embodiments, the method may (a) performing a workflow that may include a first assay and a second assay on a biological sample obtained or derived from the subject. The first assay may include a minimum residual disease (MRD) assay. A result of the workflow may be used to detect the presence or the absence of the second primary disease in the subject.

### CROSS-REFERENCE

This application is a continuation-in part of U.S. application Ser. No. 19/269,949, filed Jul. 15, 2025, which is a continuation of International Application No. PCT/IB2024/000023, filed Jan. 17, 2024, which claims the benefit of U.S. Provisional Application No. 63/439,769, filed Jan. 18, 2023, each of which are incorporated by reference herein in their entirety.

### BACKGROUND

The present disclosure relates generally to detection of a primary disease in a subject. More particularly, the present disclosure relates to detection of a second primary disease in a subject that has been previously diagnosed with a first primary disease and performing a minimal residual disease (MRD) assay of a biological sample of the subject.

After treatment for cancer, a small number of cancer cells may remain within a patient who appears to be in remission. These residual cells are called “minimal residual disease” (MRD) and may become a cause of relapse. Assays (e.g., circulating tumour DNA (ctDNA) assays) for detecting MRD can employ a variety of approaches, including sequencing a patient's tumour tissue to identify tumour-informed genetic variants, which may be indicative of MRD when detected in a patient's cell-free DNA (cfDNA).

### SUMMARY

Aspects disclosed herein provide methods for detecting a presence or an absence of a second primary disease in a subject, the method comprising: (a) performing a workflow comprising a first assay and a second assay on a biological sample obtained or derived from the subject, wherein the first assay comprises a minimum residual disease (MRD) assay, wherein the subject has been previously diagnosed with a first primary disease; and (b) using a result of the workflow to detect the presence or the absence of the second primary disease in the subject.

In some embodiments, the first assay comprises a genetic assay, and wherein the second assay comprises an epigenetic assay. In some embodiments, the first assay comprises a tumor-informed genetic assay. In some embodiments, the second assay comprises a tumor-naïve epigenetic assay. In some embodiments, the first assay or the second assay comprises a sequencing assay. In some embodiments, the first assay comprises a personalized sequencing assay, a digital signature assay, or a combination thereof. In some embodiments, the second assay comprises methylation sequencing. In some embodiments, the methylation sequencing comprises enzymatic methyl sequencing (EM-Seq), nanopore sequencing, bisulfite sequencing, or a methylation enrichment assay. In some embodiments, the second assay comprises a fragmentation assay. In some embodiments, the first assay and the second assay are performed in parallel.

In some embodiments, the workflow comprises performing a first analysis using a first signal of the first assay and performing a second analysis using a second signal of the second assay, wherein the first signal and the second signal are derived from different data, and wherein the different data are obtained from the first assay and the second assay. In some embodiments, the first assay comprises a personalized sequencing assay. In some embodiments, the first signal comprises a genetic variant signal. In some embodiments, the second assay comprises a methylation sequencing assay or a fragmentation assay.

In some embodiments, the workflow comprises performing a first analysis using a first signal of the first assay and performing a second analysis using a second signal of the second assay, and wherein the first signal and the second signal originate from different features or different measurements of the same data. In some embodiments, the same data comprises whole genome sequencing data. In some embodiments, the first assay and the second assay each comprises a digital signature assay. In some embodiments, the first signal comprises a genetic variant signal. In some embodiments, the genetic variant signal comprises a single nucleotide variant (SNV) signal. In some embodiments, the second signal comprises an epigenetic change signal. In some embodiments, the epigenetic change signal comprises a DNA methylation signal or a fragmentation signal. In some embodiments, the subject has not been diagnosed with the second primary disease. In some embodiments, the subject is asymptomatic of the second primary disease. In some embodiments, the subject is symptomatic of the second primary disease. In some embodiments, the first primary disease, or the second primary disease, or both, comprises cancer. In some embodiments, the presence of the second primary disease of the subject is detected with a sensitivity of at least 50%. In some embodiments, the absence of the second primary disease of the subject is detected with a specificity of at least 50%. In some embodiments, the biological sample comprises a blood sample, a plasma sample, or a serum sample. In some embodiments, the biological sample comprises cell-free deoxyribonucleic acid (cfDNA), cell-free ribonucleic acid (cfRNA), circulating tumor cells (CTCs), or a combination thereof.

In an aspect, described are methods of selecting genetic variants for a tumor-informed assay, the method comprising: (a) assaying a biological sample obtained or derived from a subject to generate assay results, wherein the subject has been previously identified as having a cancer; (b) generating a mutational catalogue for the assay results, wherein the mutational catalogue indicates a proportion of genetic mutation types observed in the biological sample; (c) determining a plurality of signatures associated with a type of the cancer, wherein a signature of the plurality of signatures comprises a mutational profile; and (d) determining an error of the assay results based at least on processing the mutational profile of the signature of the plurality of signatures and the assay results.

In some embodiments, the biological sample is a blood sample or a tissue sample.

In some embodiments, the biological sample is the blood sample.

In some embodiments, the blood sample is cell-free.

In some embodiments, the cancer is lung cancer, colorectal cancer, breast cancer, or skin cancer.

In some embodiments, the cancer is the lung cancer.

In some embodiments, the cancer is the colorectal cancer.

In some embodiments, the assaying comprises sequencing.

In some embodiments, the sequencing comprises whole genome sequencing or next generation sequencing.

In some embodiments, the mutational catalogue comprises one or more contexts.

In some embodiments, the one or more contexts comprises dinucleotide contexts, trinucleotide contexts, tetranucleotide contexts, pentanucleotide contexts, hexanucleotide contexts, or a combination thereof.

In some embodiments, the one or more contexts comprises two or more of the dinucleotide contexts, the trinucleotide contexts, the tetranucleotide contexts, the pentanucleotide contexts, and the hexanucleotide contexts.

In some embodiments, the one or more contexts comprises three or more of the dinucleotide contexts, the trinucleotide contexts, the tetranucleotide contexts, the pentanucleotide contexts, and the hexanucleotide contexts.

In some embodiments, the one or more contexts comprises the trinucleotide contexts.

In some embodiments, the genetic mutation types comprises single base substitutions, doublet base substitutions, insertions and deletions, copy number variations, microsatellites, or a combination thereof.

In some embodiments, the genetic mutation types comprises the single base substitutions.

In some embodiments, the genetic mutation types comprises the doublet base substitutions.

In some embodiments, the genetic mutation types comprises the insertions and deletions.

In some embodiments, the genetic mutation types comprises the copy number variations.

In some embodiments, the genetic mutation types comprises the microsatellites.

In some embodiments, the processing the mutational profile of the signature of the plurality of signatures and the assay results comprises use of a machine learning algorithm.

In some embodiments, the machine learning algorithm comprises non-negative matrix factorization (NMF), principal component analysis (PCA), or vector quantization (VQ).

In some embodiments, the machine learning algorithm comprises the NMF.

In some embodiments, the machine learning algorithm comprises the PCA.

In some embodiments, the machine learning algorithm comprises the VQ.

In some embodiments, the method further comprises applying a filtering criterion to the genetic variants, wherein the filtering criterion identifies low quality genetic variants.

In some embodiments, determining the error comprises fitting the mutational catalogue to the plurality of signatures.

In some embodiments, the fitting comprises quantifying the presence of the plurality of signatures associated with the cancer type in the mutational catalogue.

In some embodiments, determining the error comprises identifying genetic mutation types in the mutational catalogue resulting from a formalin-fixed paraffin-embedded (FFPE) process.

In some embodiments, the type of the cancer comprises a benign tumor or a malignant tumor.

In some embodiments, there is provided data analysis techniques for selecting a set of genetic variants for use in creating a tumour-informed assay. Information about genetic variants in mutational signatures for different cancer types may be used, at least in part, to filter a set of genetic variants observed in a mutational catalogue of a sample to identify genetic variants that are more likely to be attributed to mutational processes specific to the sample than other processes including, artefactual processes that result from processing the sample. The identified genetic variants can then be used to create a tumour-informed assay.

In some embodiments, a method of selecting genetic variants for a tumour-informed assay is provided. The method includes receiving a sample collected from a patient, the sample being associated with a cancer type, generating a mutational catalogue for the sample, the mutational catalogue indicating a proportion of genetic mutation types observed in the sample, selecting a set of signatures associated with the cancer type, the set including one or more signatures, each signature comprising a mutational profile, determining, based on the set of signatures associated with the cancer type and the mutational catalogue, a set of genetic variants most likely to be genuine somatic variants associated with the sample, and outputting the set of genetic variants for use in creating a tumour-informed assay for the patient.

In one aspect, selecting a set of signatures associated with the cancer type includes accessing a database configured to store a plurality of signatures associated with the cancer type, and the plurality of signatures are population-level signatures determined from a plurality of cancer samples. In another aspect, the method further includes including in the set of signatures, only signatures from the database having one or more genetic mutation types that represent at least 5% of all genetic mutations in the mutational profile. In another aspect, the method further includes including in the set of signatures, only signatures from the database having one or more genetic mutation types that represent at least 10% of all genetic mutations in the mutational profile. In another aspect, the method further includes including in the set of signatures, only signatures from the database having one or more genetic mutation types that represent at least 25% of all genetic mutations in the mutational profile.

In another aspect, determining a set of genetic variants includes fitting the mutational profiles in the set of signatures to the mutational catalogue to determine a corresponding amount that each signature is observed in the sample. In another aspect, determining a set of genetic variants further includes determining whether the amount that each signature is observed in the sample is greater than a threshold value. In another aspect, determining a set of genetic variants further includes for each of the genetic variants in the sample, associating a context probability based on a frequency of the genetic variant in the set of signatures and the determined amount that each signature is observed in the sample, and sampling the genetic variants weighted by their associated context probability to determine the genetic variants to include in the set of genetic variants. In another aspect, the context probability is based on a trinucleotide context.

In another aspect, the method further includes filtering the set of genetic variants based, at least in part, on a suitability of each of the genetic variants in the set of genetic variants for use in the tumour-informed assay, and creating the tumour-informed assay based on the set of genetic variants in the filtered set. In another aspect, the set of signatures associated with the cancer type includes at least two signatures. In another aspect, selecting the set of signatures associated with the cancer type further comprises selecting a mutational profile associated with a source of error introduced during a sequencing process, and the method further includes excluding, from the set of genetic variants, any genetic variants determined to be attributed to the source of error. In another aspect, the source of error is associated with a formalin-fixed paraffin embedded (FFPE) process, an amplification process, or a sequencing process.

In another aspect, selecting the set of signatures associated with the cancer type further comprises selecting a mutational profile associated with a therapy, and the method further includes excluding, from the set of genetic variants, any genetic variants determined to be attributed to the therapy. In another aspect, the therapy is a chemotherapy.

In another aspect, generating a mutational catalogue for the sample comprises performing whole exome sequencing on the sample. In another aspect, the set of signatures includes at least one double base substitution signature. In another aspect, at least some of the mutational profiles are associated with a different exposure type. In another aspect, the mutational profile indicates a proportion of genetic mutation types observed in a population. In another aspect, the genetic mutation types comprise a trinucleotide context. In another aspect, a signature represents an exposure to a mutational process. In another aspect, the mutational process is associated with cancer.

In some embodiments, a method of selecting genetic variants is provided. The method includes receiving a sample collected from a patient, generating a mutational catalogue from the set of genetic variants, the mutational catalogue indicating a proportion of genetic mutation types observed in the sample, selecting a set of signatures, the set including one or more signatures, each signature comprising a mutational profile, determining a set of genetic variants, excluding, from the set of genetic variants, any genetic variants determined to be attributed to the one or more signatures, and outputting the set of genetic variants for use in creating an assay for the patient.

In one aspect, the set of signatures are associated with a source of error. In another aspect, the source of error is associated with a formalin-fixed paraffin embedded (FFPE) process, an amplification process, or a sequencing process. In another aspect, the set of signatures are associated with a therapy. In another aspect, the therapy is a chemotherapy. In another aspect, the excluding further comprises excluding, from the set of genetic variants, any genetic variants determined to be sub-clonal genetic variants associated with the therapy. In another aspect, the set of signatures further comprises at least one mutational profile associated with a cancer type, wherein the determining further includes determining, based on the at least one mutational profile associated with the cancer type and the mutational catalogue, the set of genetic variants associated with the sample.

In some embodiments, a system is provided. The system includes at least one hardware computer processor programmed to perform any of the methods described herein.

In some embodiments a computer readable medium is provided. The computer readable medium is encoded with a plurality of instructions, that, when executed by at least one hardware computer processor perform any of the methods described herein.

In some embodiments, and a tumour-informed assay for monitoring the presence of cancer in a patient is provided. The tumour-informed assay is configured to detect at least one genetic variant from the output set of genetic variants according to any of the methods described herein.

### BRIEF DESCRIPTION OF DRAWINGS

Various non-limiting embodiments of the technology will be described with reference to the following figures. It should be appreciated that the figures are not necessarily drawn to scale.

FIG. 1 is an example representation of a mutational catalogue for a tumour sample that may be used to identify genetic variants for a tumour-informed assay, in accordance with some embodiments of the present disclosure.

FIG. 2 is an example representation of a single base substitution mutational signature that may be used in some embodiments of the present disclosure to identify a set of genetic variants for a tumour-informed assay.

FIG. 3 is a flowchart of a process for obtaining a set of genetic variants for use in creating a tumour-informed assay, in accordance with some embodiments of the present disclosure.

FIG. 4 is a flowchart of a process for obtaining a set of genetic variants for use in creating a tumour-informed assay, in accordance with some embodiments of the present disclosure.

FIG. 5 is an example representation of a flat mutational signature that may be present in a database of mutational signatures, in accordance with some embodiments of the present disclosure.

FIG. 6 is an example representation of selecting multiple single base substitution mutational signatures for use in a fitting process, in accordance with some embodiments of the present disclosure.

FIG. 7 is an example representation of selecting a single base substitution mutational signature and a double base substitution mutational signature for use in a fitting process, in accordance with some embodiments of the present disclosure.

FIG. 8 is an example of a process for determining whether a genetic mutation type is similar to one or more known mutational signatures, in accordance with some embodiments of the present disclosure.

FIG. 9 is an example representation of a mutational signature for an FFPE process that may be used to filter artefactual variants, in accordance with some embodiments of the present disclosure.

### DETAILED DESCRIPTION

Aspects of the technology described herein relate to techniques for determining a set of genetic variants to include in a tumour-specific assay (e.g., an MRD assay) for a patient. The inventor has recognized that at least some of the genetic variants identified through sequencing a patient's tumour tissue may be artefactual mutations caused by processes such as cytosine deamination, early incorporation errors and polymerase bias from PCR enrichment, and incorporation errors from sequencing. Including such genetic variants in a tumour-informed assay for a patient may reduce sensitivity of the assay, as they are not present in the patient's cancer. Some embodiments of the present disclosure are directed to techniques for identifying a set of genetic variants for use in a tumour-informed assay that improves the sensitivity of the assay in detecting, for example, residual cancer cells.

Some embodiments of the present disclosure relate to a method of tumour-informed variant filtering that applies mutational signature analysis to select high-quality genetic variants most likely to be present in a tumour or cancer type. Genetic mutations occur in a person's DNA through various mutational processes including, but not limited to, intrinsic errors in DNA replication, and exposure to various physical, chemical, or biological mutagens. Different mutational processes may generate particular combinations of genetic mutation types, such that a unique mutational signature may be associated with a mutational process. The existence of such mutational signatures for various mutational processes associated with cancer have recently been studied and published to publicly-accessible databases (e.g., the COSMIC database available at https://cancer.sanger.ac.uk/cosmic).

The inventors have recognized and appreciated that mutational signatures associated with different forms of cancer may be used to filter genetic variants present in a sample from a patient. The filtered set of genetic variants may be used, for example, in a tumour-informed assay for the patient. As described in more detail below, some embodiments attempt to fit genetic variants in a mutational catalogue determined for a tumour sample from a patient to a plurality of mutational signatures associated with a patient's cancer. Genetic variants associated with signatures relevant to the tumour sample (e.g., variants that fit an expected cancer type) may be prioritized for inclusion in a tumour-informed assay to improve sensitivity of the assay with respect to detecting the cancer in future samples taken from the patient. Further, by preferentially selecting genetic variants based on association with known cancer mutational signatures, no additional normal or healthy patient sample may be needed for comparison to tumour tissue to identify tumour-associated variants. The techniques described herein may also have applications in quality control, e.g., by rejecting samples that appear to be from a different kind of cancer (indicating a potential handling issue) or excluding genetic variants that appear to be associated with ex vivo mutational processes, which are unlikely to be associated with a patient's cancer, as described in more detail below.

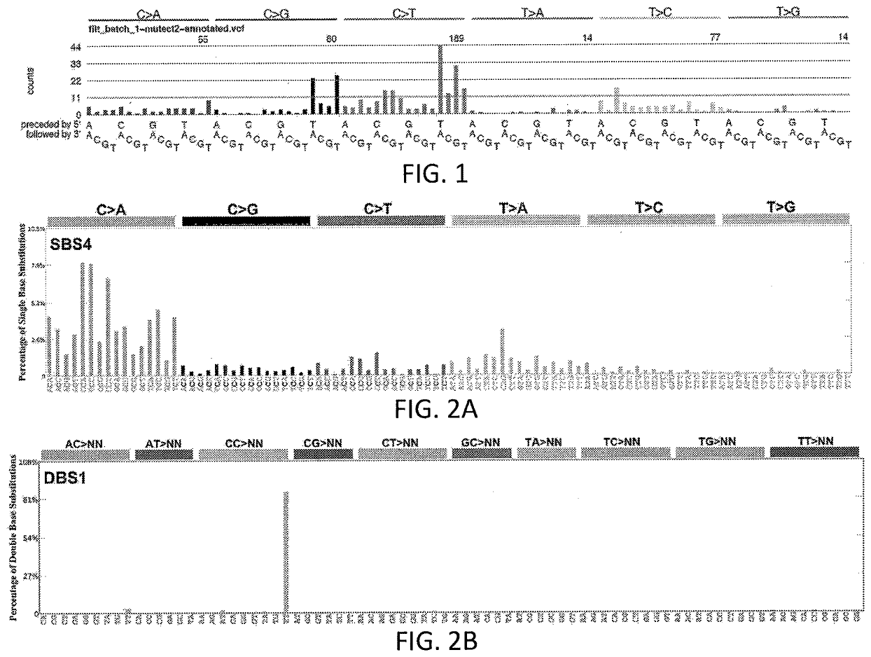

A mutational catalogue is a mutational profile that includes a set of genetic mutation types observed in a single biological sample, such as a tumour sample. In some embodiments, a mutational catalogue may include a context, such as a trinucleotide context, which identifies different combinations of nucleotides positioned at locations directly 5′ (e.g., preceding) and 3′ (e.g., following) a given mutation in a DNA sequence. FIG. 1 illustrates an example of a mutational catalogue generated for a tumour sample in accordance with some embodiments of the present disclosure. As shown, when six mutation types (C>A, C>G, C>T, T>A, T>C, T>G) and 16 trinucleotide contexts for each mutation type are considered, the mutational catalogue provides 96 “channels” of information. In some embodiments, a mutational catalogue for a tumour sample may be determined by identifying a plurality of genetic mutations relative to a reference sequence in the tumour sample and grouping the mutations according to their context. As shown in FIG. 1, a mutational catalogue may be represented as a histogram, where the heights of the bars in the histogram may correspond to either mutation counts (as shown in FIG. 1) or proportions (e.g., percentage of single base substitutions). Accordingly, a mutational catalogue for a tumour sample may indicate whether certain mutation types are over-represented or under-represented in the sample, providing insight into the types of genetic mutations most prevalent in the tumour sample.

As described above, mutational signatures for various mutational processes (e.g., mutational processes associated with cancer) have been studied and are publicly accessible in various databases (e.g., via the COSMIC database available at https://cancer.sanger.ac.uk/cosmic). Mutational signatures may be generated from a large population of samples from different individuals. For example, a mutational signature may represent recurring patterns of sequence-context-dependent genetic variants observed in patients with similar etiologies. Some unique mutational signatures associated with different cancer types and causes (e.g., exposures to different mutagens) have been identified. Mutational signatures associated with different cancer types may be generated based on whole genome sequencing (WGS) of cancer tissues from thousands of samples, with the results clustered to yield distinct mutational signatures for each cancer type. Each mutational signature may represent a landscape of both passenger and driver genetic mutations associated with that cancer. Mutational signatures may be associated with a disease (e.g., lung, colon, or skin cancer) and/or a potential cause of the disease (e.g., oxidation, microsatellite instability, exposure to ultraviolet light).

Similar to a mutational catalogue for a sample (e.g., the mutational catalogue shown in FIG. 1), a mutational signature determined based on population data can be represented as a histogram of genetic mutations. A histogram of genetic mutations for an example mutational signature (labeled SBS4) is shown in FIG. 2. The SBS4 mutational signature is associated with lung cancer from oxidation due to smoking, and is characterized by excess C>A and G>T mutations (as well as CC>AA and GG>TT double substitutions and C/G deletions, not shown). As shown in FIG. 2, the genetic mutations in a mutational signature can further be characterized by a context, such as a trinucleotide context (i.e., the different combinations of 5′ and 3′ nucleotides adjacent to any given genetic mutation). When characterized by a trinucleotide context, the characterization of a genetic mutation results in a 96-class profile for any given sample.

Although the example mutational signature shown in FIG. 2 includes genetic mutations that are single base substitutions, it should be appreciated that mutational signatures may also include types of genetic mutations other than single base substitutions including, but not limited to, doublet base substitutions, insertions and deletions, copy number variations, and microsatellites. A histogram of genetic mutations for an example mutational signature (labeled DBS1) that includes doublet-based substitutions is shown in FIG. 2. As shown, the DBS1 mutational signature, which is associated with melanoma, is characterized primarily by CC>NN mutations, and especially CC>NN mutations with a TT context (i.e., TCCT>TNNT). Other contexts for genetic variations may also be used. For example, additional nucleotides located 5′ and/or 3′ of a variation may be used to provide a dinucleotide, trinucleotide, tetranucleotide, pentanucleotide, hexanucleotide, etc. context. Insertions and deletions may be further characterized by homopolymer length, number of repeat units, and microhomology length. Copy number variations may be further characterized by length (e.g., 0-100 kb, 100 kb-1 Mb, >1 Mb, 100 kb-1 Mb, 1 Mb-10 Mb, 10 Mb-40 Mb, >40 Mb), hyperdiploid status, loss of heterozygosity, or heterozygous. Additionally, although classifying genetic variants by a context may provide additional resolution, in some embodiments, no additional nucleotide context may be used and variants may simply be binned by their base change (e.g., C->A, C->G, T->C, etc.).

As described above, mutational signatures may be generated from a large population of samples from different individuals. For instance, mutational signatures may be determined by identifying recurrent patterns in a plurality of mutational catalogues from individual patient samples. For example, consider a dataset of a large number (e.g., 1000) of mutational catalogues generated from biopsies of lung, skin, and breast cancer samples. As shown in FIG. 1, each mutational catalogue represents the counts or frequencies of different genetic mutation types (e.g., single base substitutions, double base substitutions, insertions, deletions, etc.) in association with, for example, a trinucleotide context for the genetic mutation, providing 96 different channels of information (6 mutation types across 16 different trinucleotide contexts) for the sample. By examining recurrent patterns across the mutational catalogues in the dataset (e.g., whether a C->A base change in a particular context occurs across a plurality of the samples), mutational signatures associated with different processes and/or cancer types may be identified, with each mutational signature indicating the frequency of recurrent mutation types observed in a set of samples. Any suitable pattern recognition technique may be used to identify recurrent patterns of genetic mutations in a dataset of mutational catalogues. Such techniques include, but are not limited to, machine learning techniques such as non-negative matrix factorization (NMF), principle component analysis (PCA), and vector quantization (VQ). Conventional software packages (e.g., the open source MutationalPatterns R package available at https://bioconductor.org/packages/release/bioc/html/MutationalPatterns.html) for extracting mutational signatures from a large dataset of mutational catalogues are available.

Although a user can define the parameters for extraction of mutational signatures from a mutational catalogue dataset, mutational signatures are most commonly extracted for individual cancer types (e.g., lung, breast) and/or for associated exposures (e.g., smoking, aging). Accordingly, each mutational signature extracted in this way may represent a mutational process present in a set of tumour samples. Although mutational signatures and mutational catalogues may appear to convey similar information, there are important differences. For instance, each mutational signature represents a proportion of genetic mutations across the dataset of mutational catalogues from which the signature was extracted, but includes only recurrent patterns observed in that dataset, rather than counts or proportions of all genetic mutations in a sample, as is represented in a mutational catalogue. Mutational signatures may be extracted by, for example, clustering mutational catalogues from many samples and selecting only samples with recurrent, commonly occurring profiles. An example of such mutational signature extraction is described in Degasperi et al., Substitution mutational signatures in whole-genome-sequenced cancers in the UK population, Science 376:6591(2022).

The inventor has recognized and appreciated that the genetic mutation information in mutational signatures may be leveraged to select a set of genetic variants for use in a tumour-informed assay. Accordingly, some embodiments of the present disclosure align or “fit” a mutational catalogue determined for a tumour sample to a set of mutational signatures, which enables a determination of which mutational processes (as represented in the signatures) are most closely associated with the sample.

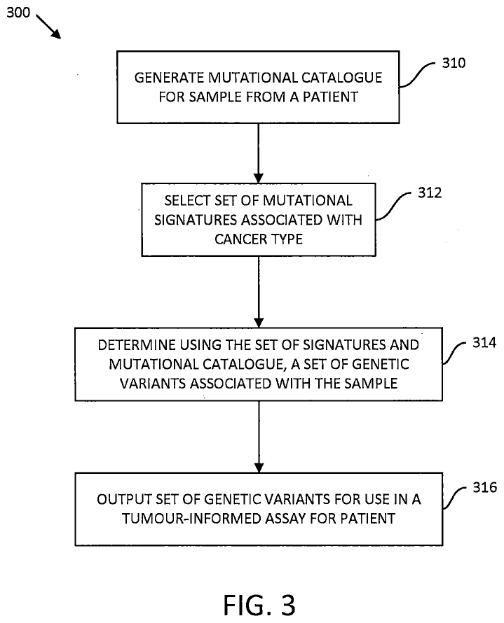

FIG. 3 illustrates a process 300 for determining a set of genetic variants for use in a tumour-informed assay, in accordance with some embodiments of the present disclosure. In act 310, a mutational catalogue for a sample from a patient is generated. For example, as described in more detail below, a sample may be processed and DNA sequenced to generate a text-based file that describes the genetic mutations in the sample relative to a reference genetic profile. The occurrence of genetic mutations and, optionally, their context may be formulated as a mutational catalogue for the sample. Process 300 then proceeds to act 312, where a set of mutational signatures associated with a particular cancer relevant to the sample are selected. For instance, the COSMIC database v.3.3 from June 2022 includes approximately 130 different mutational signatures extracted from mutational catalogues for a large population of tumour samples for different cancers. In act 312, a set of mutational signatures likely to be most relevant to the particular cancer associated with the sample analyzed in act 310 may be selected.

Process 300 then proceeds to act 314, where a set of genetic variants most likely to be genuine somatic variations associated with the sample is determined using the mutational catalogue generated in act 310 and the set of mutational signatures in the set selected in act 312. For instance, as described in more detail below, the mutational catalogue for the sample may be fit to the set of mutational signatures to determine a weighted sum of mutational signatures that represent the most likely exposures resulting in the mutational catalogue. As an example, a mutational catalogue from a lung cancer patient may include mutations associated with aging (e.g., C->T changes from deamination), smoking, and other mutational processes. Due to different amounts of exposure to these mutational processes, different genetic mutations associated with the signatures may be present in some lung cancer samples but not others. Fitting a mutational catalogue to a set of mutational signatures, as described herein may deconvolute the mutational processes active in a tumour for a particular patient. By fitting the mutational catalogue to the set of mutational signatures, each genetic variant in the mutational catalogue may be associated with a particular mutational signature, providing insight into the mutational processes associated with the evolution of that tumour sample.

After the set of genetic variants that characterize the sample are determined in act 314, process 300 proceeds to act 316, where the set of genetic variants is output for use in creating a tumour-informed assay for the patient. By detecting which genetic variants are likely to be associated with cancer rather than being artefactual, and also selecting variants that characterize the underlying mutation processes associated with the sample, an assay that is sensitive to those genetic variants may be generated.

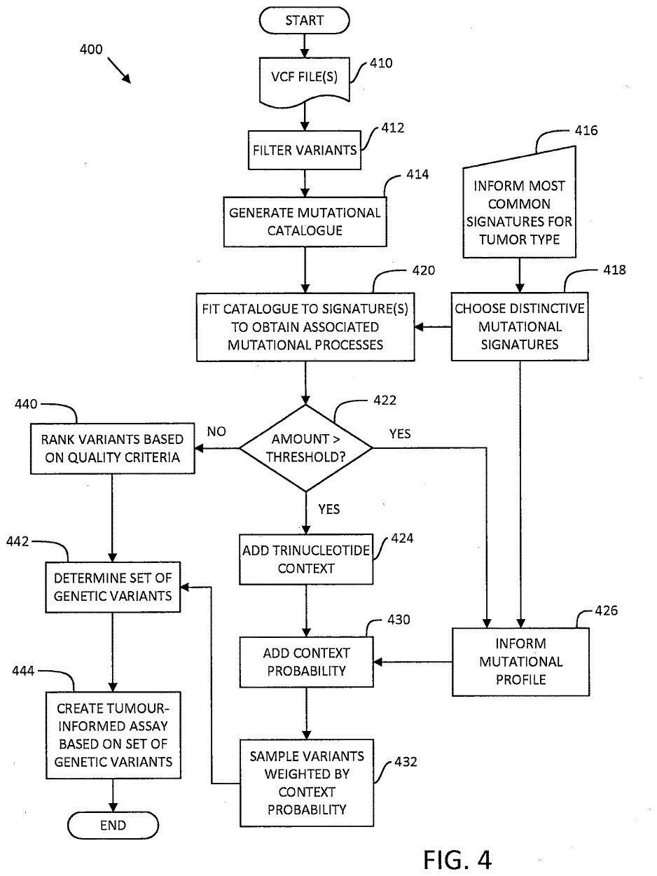

FIG. 4 illustrates a process 400 for creating a tumour-informed assay based on a set of genetic variants selected in accordance with the techniques described herein. In act 410, a sample (e.g., a blood sample, tumour biopsy, or other tissue sample) may be processed (e.g., using whole exome sequencing or another next generation sequencing technique) to determine genetic sequence variations in the sample relative to a reference genome, with the output being a text-based variant call format (VCF) file. Process 400 may then proceed to act 412, where the genetic variants identified in the VCF file may be optionally subjected to one or more filtering criteria to identify low-quality genetic variants, such as genetic variants with low coverage, low base or mapping qualities, redundant surrounding sequence, presence in online databases (e.g., has a frequency >0.05 in dbSNP indicating that it is likely a germline variant), and the like. Process 400 then proceeds to act 414, where a mutational catalogue for the sample is generated using the genetic variants identified in the VCF file that were not filtered out in act 412. For instance, a matrix including the genetic variants in the mutational profile of the sample and their counts or proportions, may be generated as the mutational catalogue for the sample. An example mutational catalogue for a sample is shown in FIG. 1, described above. Although filtering low-quality variants may be optional in some embodiments, of the present disclosure, it may be useful to remove these variants as they may impact the signature fitting process described herein.

Process 400 then proceeds to act 420, where the mutational catalogue for the sample is fit to a set of one or more mutational signatures. As described above, fitting the mutational catalogue to one or more mutational signatures attempts to uncover the underlying mutational process(es) to which the sample was subjected by mapping variants in the mutational catalogue for the sample to variants in known genetic profiles for different mutational processes. For example, consider a mutational catalogue containing 1,000 mutations. After fitting in act 420, 500 mutations may be attributed to the SBS4 signature (smoking), 200 mutations may be attributed to SBS3 (defective homologous recombination DNA damage repair), and the remainder of the mutations may be associated with other signatures, or no signature at all. Each attribution to a particular signature may be considered an “exposure” to the process associated with that signature. In this way, fitting a mutational catalogue for a sample from a patient to a set of population-based signatures in accordance with some embodiments of the present disclosure may show that the patient has been exposed to certain mutational processes as defined by certain cancer signatures.

Fitting techniques attempt to determine the contribution of each mutational process to the genetic variants expressed in a mutational catalogue by quantifying the presence and prevalence of each mutational signature in the mutational catalogue. In some embodiments, fitting may be performed by creating two matrices-a sample matrix M and a signatures matrix P. The sample matrix M may include 96 rows for each mutation/trinucleotide context combination and n columns for each mutational catalogue. The signatures matrix P may include k rows for each mutational signature and 96 columns for each trinucleotide context. Given these two matrices, a weights matrix E including k columns for each signature and n rows for each sample may be determined such that a reconstructed tumour sample matrix R (computed as M−(P*E)) minimizes a given error threshold e. Stated differently, the weights matrix E may be determined such that the matrix E best recreates M, by finding the best combination of exposures that minimize the differences between (P*E) and M. For a given sample n, the highest weights for the corresponding row in E may be selected to understand which mutational process was most likely to result in each mutation type in the mutational catalogue. It should be appreciated that the other techniques including, but not limited to, other minimization techniques, golden search, minimal quadratic, and the like, may alternatively be used to fit a mutational catalogue to a set of mutational signatures in act 420.

The inventors have recognized and appreciated, that the accuracy of genetic variants output from the fitting process in act 420 depends considerably on which mutational signatures are provided as input to the fitting process. Accordingly, some embodiments of the present disclosure include one or more signature curation acts to select from a database of signatures, a set of signatures for use in the fitting act 420. As shown in FIG. 4, the signature curation acts may include an act 416 of selecting signatures that are most common for or are otherwise expected to be observed in the particular cancer type associated with the sample. For instance, the inventors have recognized that, due to the sensitivity of the fitting algorithm(s), fitting a mutational catalogue to mutational signatures associated with mutational processes that are not related to, for example, the particular cancer type associated with the sample, may implicate mutational processes in the fitting results that are less likely to have occurred for the particular patient. In some cases, fitting algorithms may force the fitting of certain variants to a signature even if the sample was not exposed to that mutational process. As such, the accuracy of genetic variants output from the fitting process in act 420 may be improved by pre-selecting certain signatures that are expected to be observed in such a sample.

In one example, the “smoking signature” SBS4, shown in FIG. 2 is found in a variety of tumour types, even when it is unlikely that tobacco carcinogens would reach that site (such as in prostate cancer). This may be because the SBS4 signature is dominated by C>A and G>T mutations which are found in many cancers. Accordingly, if the SBS4signature was included in the set of mutational signatures used to analyze a prostate cancer sample, the fitting process in act 420 may preferentially fit variants to SBS4 instead of other signatures due to this strong phenotype, even though the patient may never have smoked. Similarly, mutational signatures which are associated with mutational processes common across all types of samples may not yield as meaningful results in the fitting process than mutational signatures that are associated with a more specific process that is not common across all or many types of samples. For instance, the SBS1 mutational signature (aging related, and thus not exclusive to somatic changes), as well as the SBS3, SBS5, and SBS8 mutational signatures, which include common mutations across all cancer types may not be preferred candidates for use in the fitting process of act 420.

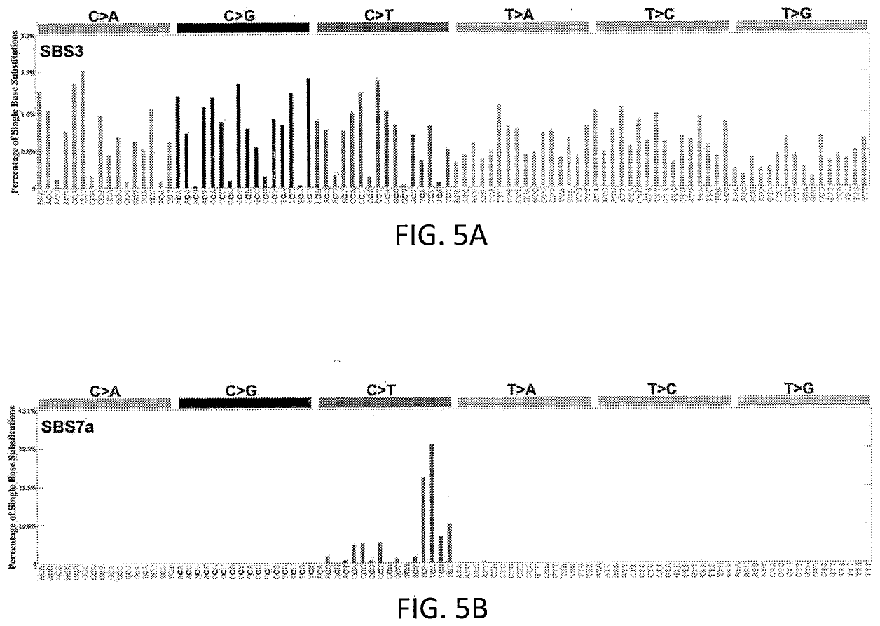

The inventors have also recognized that fitting techniques that use minimization tend to prefer “flat” signatures in which mutation types are evenly distributed. An example of a flat signature (labeled SBS3) is shown in FIG. 5. As can be observed in FIG. 5, each mutation type in the SBS3 signature has a similar contribution to the overall mutational profile (e.g., none of the mutation types exceeds a frequency of 1%, 2%, 2.5%, 3%, 3.5%, 4% or 4.5%). If included in the set of signatures provided as input to the fitting process in act 420, many or all variants in many mutational catalogues may become associated with this flat signature, resulting in an overfitting to the signature. In an effort to address the inclusion of such flat signatures, in some embodiments of the present disclosure, only signatures that are “distinctive” may be included in the set of signatures provided as input to the fitting process in act 420, as shown in act 418 of process 400. An example of a mutational signature (labeled SBS7a) that may be considered distinctive is shown in FIG. 5. As shown, the SBS7a signature shows a clear preference for a particular mutation type (in this case C>T substitutions) relative to other mutation types. Other non-limiting examples of distinctive mutational signatures include SBS2 (activity of APOBEC family of cytidine deaminases) and SBS10d (defective POLDI proofreading).

In some embodiments, flat signatures may be identified visually, empirically (e.g., by fitting multiple mutational catalogues to a signature and noting that the signature tends to be selected for most variants across the multiple mutational catalogues), or by setting a threshold for the proportion of contributions for a given mutation type needed for inclusion. For example, in some embodiments, to be considered a distinctive signature (as opposed to a flat signature), the signature may have a given mutation type that represents at least 5% of all genetic mutations in the signature's mutation profile. In some embodiments, the threshold may be at least 10%, at least 15%, at least 20%, at least 25%, at least 50%, or at least 75% of all genetic mutations in the signature's mutation profile.

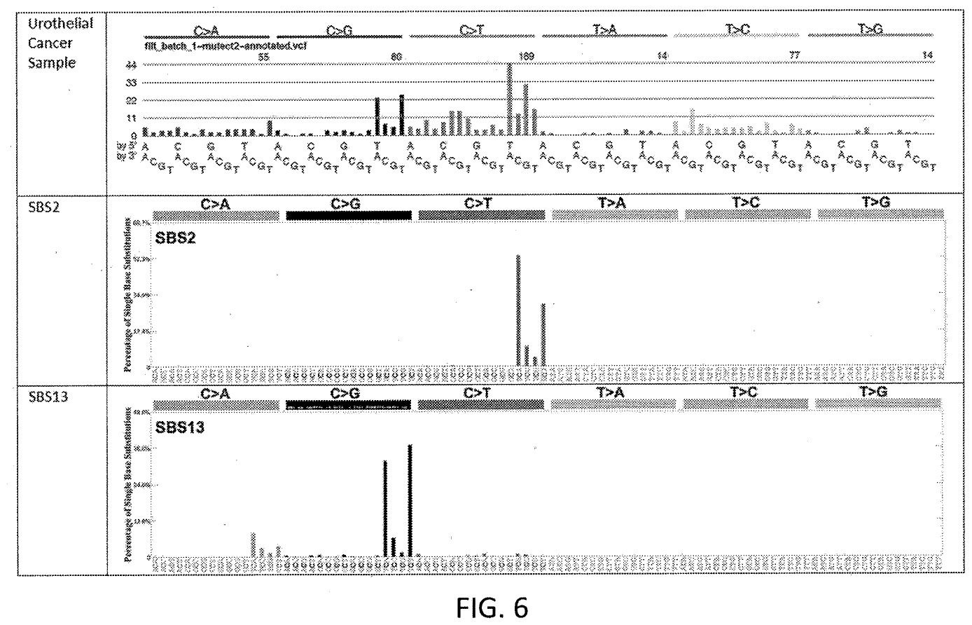

An example of selecting distinctive mutational signatures for use in the fitting process of act 420 in FIG. 4 is illustrated in FIG. 6. In the example of FIG. 6, the tumour sample is a urothelial cancer sample. In act 416 of process 400, it is determined that four mutational signatures are often observed for this cancer type: SBS1, SBS13, SBS2, and SBS5. Although each of these signatures is relevant to urothelial cancer, SBS5 may be characterized as a flat (e.g., not distinctive) signature and may be removed in act 418 of process 400 from the set of signatures used in the fitting process because it is not distinctive, and SBS1 may be removed in act 416 of process 400 because it is observed in many cancer types and thus may not be informative. Thus, the resulting set of mutational signatures used for fitting includes only the SBS2 and SBS13 signatures as shown in FIG. 6. A visual comparison of the SBS2 and SBS13 mutational signatures with the mutational catalogue of the tumour sample confirms that the mutational profiles of the SBS2 and SBS13 signatures include relevant genetic mutations to characterize the tumour sample and also have a strong mutational phenotype. In particular, FIG. 6 shows that the tumour sample is essentially a combination of the C>T mutational profile of SBS2 and the C>G mutational profile of SBS13, though the proportions differ. This may be due to the mutational catalogue representing the amount of exposure the sample has received from each of the mutational processes associated with SBS2 and SBS13. In the example of FIG. 6, nearly 80% of the mutational catalogue for the sample can be explained by the SBS2 and SBS13 signatures, of which 60% is a result of exposure with SBS2 and 40% is a result of exposure with SBS13.

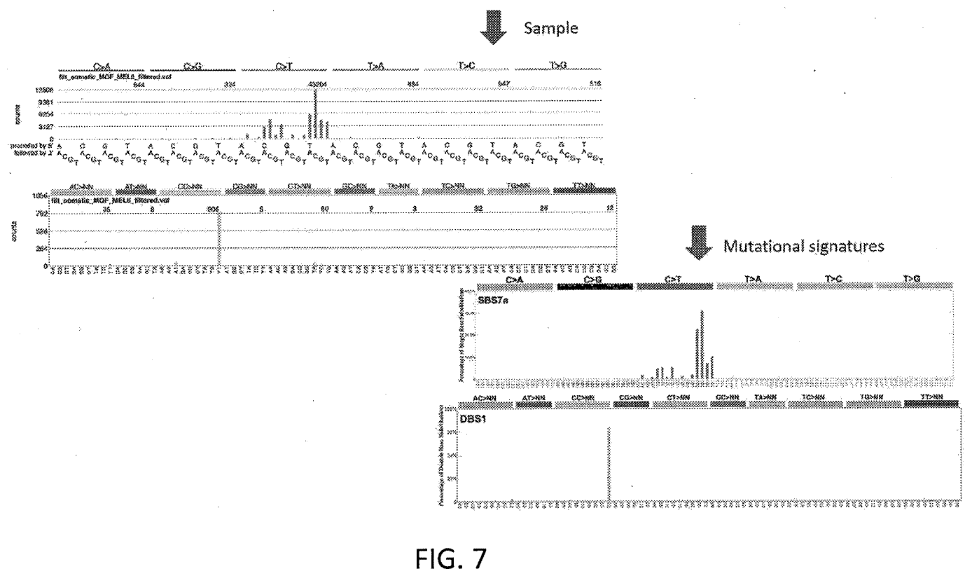

Another example of selecting distinctive mutational signatures for use in the fitting process of act 420 in FIG. 4 is illustrated in FIG. 7. In the example of FIG. 7, the tumour sample is a melanoma sample. As shown in FIG. 7, the mutational catalogue for the melanoma sample includes both single base substitutions (top) and doublet base substitutions (bottom). It may be determined that one distinctive single base substitution cancer mutational signature (SBS7a) and one distinctive doublet base substitution cancer mutational signature (DBS1) are often observed for the melanoma cancer type, and this set of mutational signatures may be used for fitting in act 420 of process 400 shown in FIG. 4. A visual comparison of the SBS7a and DBS1 mutational signatures with the mutational catalogue of the melanoma tumour sample confirms that the mutational profiles of the SBS7a and DBS1 signatures include relevant genetic mutations to characterize the tumour sample.

In some embodiments, the number of signatures used in the fitting process is between 2 and 5 signatures. In some instances, using additional signatures may impact the results of the fitting process, e.g. by overfitting.

Returning to process 400 shown in FIG. 4, after fitting is complete in act 420, process 400 proceeds to act 422, where it may be determined whether the exposure of the sample to a particular signature determined during the fitting process is above a threshold. If it is determined in act 422, that the sample has an exposure amount to at least one signature above the threshold, process 400 proceeds to act 424, where a context (e.g., a trinucleotide context) is added to each variant in the set of variants. This context may be used to assign a context probability (act 430) from the relevant subset of signatures identified in act 422 (act 426), such that each variant is annotated with its likelihood of arising due to an exposure from one of the cancer mutational signatures used for the fitting process. For example, in the urothelial cancer sample example of FIG. 6, the SBS13 signature, C>A (TCC context) has a lower proportion than C>G (TCA context). Accordingly, C>G (TCA) changes in the mutational catalogue may be assumed to have a higher probability of being associated with urothelial cancer than C>A (TCC) changes. In some embodiments, the context probability may be determined as the sum of the associated exposures. For example, if it is determined that 60% of the C>T(TCA) variant is a result of exposure to SBS2 and 40% is a result of exposure to SBS13, then the context probability for C>T (TCA) can be calculated as the sum of 0.6*C>T(TCA) frequency in SBS2 and 0.4*C>T (TCA) frequency in SBS13, adjusting for the determined amount of exposure to each cancer mutational signature.

After assigning context probabilities to the variants in act 430, process 400 proceeds to act 432, where the variants are sampled based on the assigned context probabilities to yield a set of genetic variants in act 442 most likely to be associated with the tumour sample. In particular, sampling may yield genetic variations that correspond to the peaks in the mutational signatures. Alternatively, the genetic variants may be ranked based on the context probabilities and a top number (e.g., 6, 12, 16, 20, 48, 96) may be chosen for inclusion in the set of variants determined in act 442. By considering mutational processes likely to be relevant to a tumour development and enabling a probabilistic association of the variants to these processes, the variants selected are more likely to be real somatic changes as opposed to variants introduced by artefactual processes.

If it is determined in act 422 that the fitting process did not identify any exposure amounts greater than a threshold value, process 400 may proceed to act 440, where the variants included in the VCF file are instead ranked based on one or more quality criteria. For example, variants may be ranked based on the depth of coverage in a normal or healthy sample, the number of reads between tumour and normal samples, allele frequencies, the probability of a variant being a germline variant, and the like. Additional examples of such quality criteria include may be found in International Patent Publication WO2022029688A1, the contents of which is hereby incorporated by reference. The ranked variants may then be used to determine the set of genetic variants in act 442 that may be used to create a tumour-informed assay.

In some embodiments, variants may also be weighted based on their possibility of being a driver mutation (i.e., a mutation likely to be associated with causing cancer) or a passenger mutation. The latter may be particularly useful for identifying minimal residual disease as they are less likely to be extinguished by targeted treatments, and thus may still be useful for identifying resistant clones that have lost driver mutations. In some embodiments, variants may also be weighted or ranked on other features such as clonality (i.e., clonal or subclonal), mappability, or likelihood of being amplified, as further described in International Patent Publication WO2022029688A1.

After the set of genetic variants is determined in act 442 (e.g., either through ranking the variants in act 440 or sampling the variants based on context probability in act 432), process 400 proceeds to act 444, where the set of variants may be used to create a tumour-informed assay. The tumour-informed assay may be designed to preferentially amplify the set of variants in a subsequent plasma sample to detect residual disease. Examples of tumour-informed assays may be found, for example, in International Patent Publication WO 2022-029688A1 and US Patent Publication 2020/0157604, the contents of which are hereby incorporated by reference.

Although the techniques described herein may prioritize cancer-associated variants based on context (e.g., by sampling variants based on context probability in act 432 of process 400), some artefactual variants may still be selected for inclusion in an assay (though typically they may be prioritized lower than the cancer-based variants). In such cases, it may be advantageous to specifically exclude any variants that are not associated with any previously seen cancer signature, due to the low likelihood that there would be a genetic variant observed in a mutational catalogue that has not been seen before in any of the known cancer mutational signatures. Filtering out likely artefactual variants in a mutational catalogue may be performed by comparing each variant in the mutational catalogue to various known cancer signatures via, e.g., cosine similarity. In some embodiments, all available mutational signatures (e.g., not restricted to a particular cancer type) may be used for the artefactual variant filtering, as the filtering may determine whether a given mutational profile is similar to any mutational process (e.g., signature) that has been previously observed. Any variants that are not similar to a previously observed signature are likely to be a result of some other process introducing error, which may be due to, for example, to sample preparation or amplification. Returning to the urothelial cancer sample example in FIG. 6, the artefactual variant filtering process would likely not exclude the C->T and C->G variations, as these mutational profiles are present in the SBS2 and SBS13 mutational signatures, respectively. However, other variants (e.g., the T->C variations) may be excluded if they are not sufficiently similar to some other mutational process observed in a mutational signature.

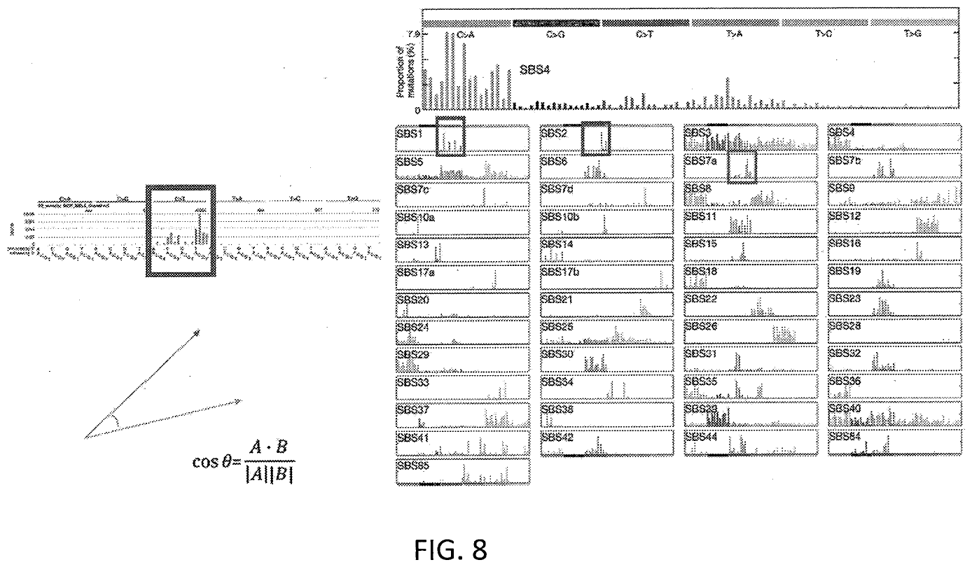

In some embodiments, rather than analyzing all variants in a mutational catalogue, samples showing an excess (e.g., >50%, >60%, >70%, >80%) of mutations for a particular mutational type (e.g., C>T, including all contexts) may be flagged for further analysis. The mutational profile for a given flagged mutation may be compared with all (or a subset) of mutational signatures also showing a significant proportion for this mutation type using, e.g., cosine similarity, an example of which is shown in FIG. 8. If the mutation profile is too dissimilar to any of the signatures, the mutation type may be removed from further consideration of being included in the final set of genetic variants. As shown in the example of FIG. 8, a sample having excess C>T mutations may be flagged and then compared pairwise with a plurality of mutational signatures. Using cosine similarity, the mutational profile for C>T mutations may be determined to be sufficiently similar (e.g., using cosine similarity and a threshold of 0.85) to corresponding mutational profiles from within the SBS1, SBS2, and SBS7a mutational signatures. Accordingly, this variant type may be retained for further consideration in the variant selection process.

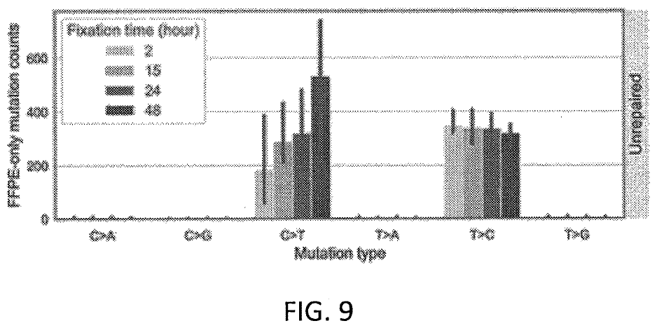

In some embodiments of the present disclosure, artefactual variants in the mutational catalogue may be identified and removed by comparison of the variant to an “artefactual signature” generated from mutational catalogues corresponding to samples containing many artefactual mutations. For example, variations resulting from the formalin-fixed paraffin-embedded (FFPE) process can be identified and removed based on their similarity to FFPE mutational signatures. FIG. 9 show an example of an artefactual signature for FFPE-based mutations. Such a signature may be used to identify and exclude certain variants in the mutational catalogue that are likely due to the FFPE process rather than being due to exposure to a mutational process for the particular type of cancer associated with the sample. Similarly, artefactual signatures can be generated from samples with PCR amplification errors or other error types to provide a filter for excluding certain variants from a mutational catalogue.

In some embodiments, a set of mutational signatures includes at least one mutational signature associated with exposure to a therapy, such as chemotherapy. Some mutational exposures happen earlier in cancer development than others, and therefore some signatures are more likely to represent clonal variations whilst others are more likely to be sub-clonal variations (i.e., the signatures associated to the earlier exposures are more likely to be clonal whilst signatures associated with certain later events such as chemotherapy will be sub clonal). Using this information allows for the selection of variants that are more likely to be clonal. This has value, for example, when a patient has already had some treatment (e.g. a breast cancer sample taken after neoadjuvant therapy). Variations from the tumour can be prioritized based on the signatures by selecting variants that are: a) more likely to be clonal, and b) more likely to be real somatic changes. Similarly, variants may be excluded if they are more likely to be clonal.

Having thus described several aspects and embodiments of the technology set forth in the disclosure, it is to be appreciated that various alterations, modifications, and improvements will readily occur to those skilled in the art.

Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more hard drives, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,”“holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of selecting genetic variants for a tumor-informed assay, the method comprising:
(a) assaying a biological sample obtained or derived from a subject to generate assay results, wherein the subject has been previously identified as having a cancer;
(b) generating a mutational catalogue for the assay results, wherein the mutational catalogue indicates a proportion of genetic mutation types observed in the biological sample;
(c) determining a plurality of signatures associated with a type of the cancer, wherein a signature of the plurality of signatures comprises a mutational profile; and
(d) determining an error of the assay results based at least in part on processing the mutational profile of the signature of the plurality of signatures and the assay results.

2. The method of claim 1, wherein the biological sample is a blood sample or a tissue sample.

3. The method of claim 2, wherein the biological sample is the blood sample.

4. The method of claim 3, wherein the blood sample is cell-free.

5. The method of claim 1, wherein the cancer is lung cancer, colorectal cancer, breast cancer, or skin cancer.

6. The method of claim 5, wherein the cancer is the lung cancer.

7. The method of claim 5, wherein the cancer is the colorectal cancer.

8. The method of claim 1, wherein the assaying comprises sequencing.

9. The method of claim 8, wherein the sequencing comprises whole genome sequencing or next generation sequencing.

10. The method of claim 1, wherein the mutational catalogue comprises one or more contexts.

11. The method of claim 10, wherein the one or more contexts comprises dinucleotide contexts, trinucleotide contexts, tetranucleotide contexts, pentanucleotide contexts, hexanucleotide contexts, or a combination thereof.

12. The method of claim 11, wherein the one or more contexts comprises two or more of the dinucleotide contexts, the trinucleotide contexts, the tetranucleotide contexts, the pentanucleotide contexts, and the hexanucleotide contexts.

13. The method of claim 11, wherein the one or more contexts comprises three or more of the dinucleotide contexts, the trinucleotide contexts, the tetranucleotide contexts, the pentanucleotide contexts, and the hexanucleotide contexts.

14. The method of claim 11, wherein the one or more contexts comprises the trinucleotide contexts.

15. The method of claim 1, wherein the genetic mutation types comprises single base substitutions, doublet base substitutions, insertions and deletions, copy number variations, microsatellites, or a combination thereof.

16. The method of claim 15, wherein the genetic mutation types comprises the single base substitutions.

17. The method of claim 15, wherein the genetic mutation types comprises the doublet base substitutions.

18. The method of claim 15, wherein the genetic mutation types comprises the insertions and deletions.

19. The method of claim 15, wherein the genetic mutation types comprises the copy number variations.

20. The method of claim 15, wherein the genetic mutation types comprises the microsatellites.

21. The method of claim 1, wherein the processing the mutational profile of the signature of the plurality of signatures and the assay results comprises use of a machine learning algorithm.

22. The method of claim 21, wherein the machine learning algorithm comprises non-negative matrix factorization (NMF), principal component analysis (PCA), or vector quantization (VQ).

23. The method of claim 22, wherein the machine learning algorithm comprises the NMF.

24. The method of claim 22, wherein the machine learning algorithm comprises the PCA.

25. The method of claim 22, wherein the machine learning algorithm comprises the VQ.

26. The method of claim 1, further comprising applying a filtering criterion to the genetic variants, wherein the filtering criterion identifies low quality genetic variants.

27. The method of claim 1, wherein determining the error comprises fitting the mutational catalogue to the plurality of signatures.

28. The method of claim 27, wherein the fitting comprises quantifying the presence of the plurality of signatures associated with the cancer type in the mutational catalogue.

29. The method of claim 1, wherein determining the error comprises identifying genetic mutation types in the mutational catalogue resulting from a formalin-fixed paraffin-embedded (FFPE) process.

30. The method of claim 1, wherein the type of the cancer comprises a benign tumor or a malignant tumor.
